(19) United States
(12) Patent Application Publication
(10) Pub. No.: US 2026/0297005 A1
(43) Pub. Date: Oct. 1, 2026

(54) Method for Producing Odorants and Fragrances on a Thin-Film Evaporator

(71) Applicant: Symrise AG, Holzminden (DE)

(72) Inventors: Bernd HÖLSCHER, Halle (DE); Frank GERSTMANN, Frankfurt am Main (DE); Stefanie BARGSTEN, Steinheim (DE); Gennadiy KOLOMEYER, Jacksonville, FL (US); Emilie SINGER, Holzminden (DE)

(21) Appl. No.: 18/879,668

(22) PCT Filed: Aug. 22, 2022
(86) PCT No.: PCT/EP2022/073310

§ 371 Date: Dec. 27, 2024

Related U.S. Application Data
(60) Provisional application No. 63/357,164, filed on Jun. 30, 2022.

(51) Int. Cl. C07C 45/51; C07C 45/82

(52) U.S. Cl. CPC C07C 45/515; C07C 45/82

(57) ABSTRACT

The present invention relates to a process for the preparation of an odorant or fragrance by carrying out thermal rearrangement reactions of chemical compounds under mild and resource-saving conditions in a thin-film evaporator under distillative conditions, as well as to the products, i.e. compounds directly resulting from this process. In particular, the present invention relates to an alternative and novel process for producing the odorant of 3-(4-isopropylcyclohexen-1-yl) propanal via a thermal rearrangement reaction in a thin film evaporator.

### FIELD OF THE INVENTION

The present invention relates to a process for the preparation of perfumes or fragrances by carrying out thermal rearrangement reactions of chemical compounds under mild and resource-saving conditions in a thin-film evaporator under distillative conditions, as well as to the products, i.e. compounds directly resulting from this process. In particular, the present invention relates to an alternative and novel process for the preparation of 3-(4-isopropylcyclohexen-1-yl) propanal of formula (I).

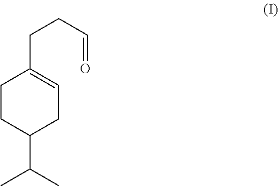

### STATE OF THE ART

Primary requirements for manufacturing methods for chemical products are cost-effectiveness, high yield and selectivity while complying with all safety-relevant aspects. A material-efficient and therefore cost-efficient production provides a considerable competitive advantage over competitors, particularly in view of the increasing demand for certain chemical products, such as fragrances and perfumes. In this context, it should be borne in mind that the efficient use of materials reduces production costs and can also increase the productivity of the manufacturing process at the same time.

Optimizing the use of materials in production while taking environmental aspects into account is therefore a major challenge in the manufacture of chemical compounds and their further processing as raw materials, chemical products, ingredients or similar.

In this context, chemical manufacturing processes should also consider aspects of so-called green chemistry, i.e. environmental friendliness.

Another key aspect is therefore to make manufacturing processes as material-efficient as possible by using recycled residual materials, unconverted materials, waste materials or by-products as secondary raw materials for further processes or, ideally, as starting materials for the underlying process as such, thereby protecting the environment and reducing production and disposal costs at the same time. Reusing resources in this way conserves existing stocks and thus makes a decisive contribution to the sustainability of the manufacturing process, while at the same time reducing the costs incurred.

In addition, mild reaction conditions are preferable due to the low energy input and often lead to more controllable and uniform selectivities of the products in chemical processes.

Temperature-sensitive substances, such as many fragrances and/or flavorings, may only be heated to high temperatures for a short time in order to counteract unwanted thermal decomposition processes. Classic distillation processes generally lead to long thermal stress on the component to be distilled. This can have negative effects on the yield as well as on the quality of the desired chemical product, provided it has thermolabile properties. Fast distillation processes with satisfactory separation performance and short residence times are therefore desirable.

Fast reactions and therefore short residence times can increase the space-time yield and reduce the thermal stress on chemical products, which often has a negative impact on the quality and properties of the products. As already mentioned, reasons for this can be, for example, thermal decomposition due to excessive thermal stress.

One way of distilling large quantities of starting material within short residence times without exposing it to excessive thermal stress is now to use thin-film evaporators, in which the material to be processed is applied as a thin film to the inner surface of the evaporator, while only very short contact times with the heated surface exist.

Thin film evaporators are primarily suitable for the thermal separation of a mixture of substances by distillation, so thin film evaporators are used in the purification of mixtures of substances, among other things.

For example, EP 3103538 A1 describes a thin-film evaporator for the thermal separation of readily volatile fractions from heavier boiling residues in a mixture of substances by evaporation, which has a closed, essentially cylindrical reactor vessel, the reactor wall of which can be heated by means of a heating device, with an inlet for the feed material to be separated, an outlet for the residues and an outlet for the vapors, and with at least one drive shaft arrangement connected to a drive means, which has at least one wiping device with a rotor with wiper elements for the inner surface of the reactor wall.

The cosmetics industry, perfumery and similar sectors of the economy, for example, have an increasing demand for high-quality ingredients and active substances. Such substances can be aroma and fragrance or odiferous substances, for example. Since fragrances often have very specific odor profiles, it is therefore particularly desirable to synthesize such products as pure and free of impurities as possible, i.e. as selectively as possible, since potential impurities in the fragrance can distort or adversely affect the characteristic odor impression and cause unpleasant secondary odors. Such impurities can also have a negative effect on the stability of the synthesized fragrances due to chemical interactions and cause unpleasant odor notes or change the underlying odor impression. The highest possible degree of purity of such compounds is therefore desirable.

Although mixtures of substances can currently be freed from unwanted by-products, reaction educts or degradation products by means of distillation, e.g. in a thin-film evaporator on a large scale, as mentioned above, the current manufacturing processes are associated with high costs, low selectivities and low material efficiency.

Thermal rearrangement reactions often form the basis of many synthetic processes and often only take place under harsh thermal conditions. However, such high thermal stresses often lead to thermal decomposition of the resulting compounds or corresponding by-products, which can interact adversely with the products. As a result, sufficient selectivity of production and therefore good product quality can no longer be guaranteed. For example, the synthesis of 3-(4-isopropylcyclohexen-1-yl) propanal, as already described in EP 2578671 A1, is carried out via a thermally driven Claisen rearrangement. However, a low selectivity of the reaction can be observed, as the by-product 5-isopropyl-2-methylenecyclohexanol is also formed during the acidic cleavage of the acetal.

Document EP 2247648 B1, for example, describes a process for the production of lactamates by thin-film evaporation by reacting alcoholates with lactams. In this process, a reaction of the alcoholate with the lactam takes place on the thin-film evaporator to form the catalytically active lactamate. This reaction releases the corresponding alcohol, which is then removed from the reaction mixture directly on the thin film evaporator.

Patent document WO 2005030358 A1 discloses a thin film evaporator designed to increase the separation efficiency and, if necessary, to carry out or accelerate chemical reactions during the evaporation process. For this purpose, in a preferred embodiment, the interior of the thin film evaporator is designed as a catalyst. Tests have shown that a combination of distillation, absorption and chemical reaction can be carried out by installing heterogeneous catalysts in the evaporation chamber and/or by installing mass transfer surfaces and by adding reactants directly into the evaporation chamber.

U.S. Pat. No. 5,561,209 A discloses a continuous process for the preparation of polyorganosiloxanes by condensation reaction of low molecular weight polyorganosiloxanols with each other or with oligomeric siloxanes in the presence of a catalyst system in a thin film evaporator.

A continuous process for the production of the fragrance citral is disclosed in WO 2008037693 A1. In particular, the document deals with the production of the corresponding acetals. The apparatus described therein for producing the unsaturated acetals preferably comprises a distillation column which is used as a reaction column, the citral formed being continuously removed from the reaction mixture by distillation during the reaction.

An alternative continuous process for the production of citral by thermal cracking of 3-methyl-2-butene-1-al-diprenyl acetal is described in EP 0992477 B1. The thermal cracking of the acetal is carried out in the lower part or in the bottom of a distillation column with 5 to 100 theoretical separation stages.

However, none of the cited prior art documents describe the production of fragrances or odiferous substances by carrying out thermal rearrangement reactions under controlled and material-efficient conditions in a thin-film evaporator. The high effectiveness of the process described therein is due to mild reaction conditions, which avoid thermal stress on the products and side reactions.

The invention is thus based on the general task of providing a process in which fragrances or odorants can be produced efficiently and gently.

A further task of the invention relates to a highly selective process for the preparation of 3-(4-isopropylcyclohexen-1-yl) propanal.

Therefore, the present invention relates to an efficient production process in combination with effective purification of the produced fragrances or odiferous substances in a single combined process step.

From a further point of view, the present invention also relates to the material- and cost-efficient production of fragrances or odiferous substances, taking into account environmental aspects.

### SUMMARY OF THE INVENTION

The present problem is solved by the objects of the independent patent claims. Preferred embodiments result from the wording of the dependent patent claims and the following description and examples of embodiments.

In a first aspect, the present invention relates to a process for producing a fragrance or odorant by carrying out thermal rearrangement reactions comprising the following steps:
a) Providing at least one reaction educt and/or other reagents in a thin film evaporator;
b) Carrying out at least one thermal rearrangement reaction on the reaction educts from step a) in the thin film evaporator under distillative conditions;
c) Obtaining the fragrance or odorant.

In a next preferred variant of the process described above, the product of step c) is the compound 3-(4-isopropylcyclohexen-1-yl) propanal. Thus, a preferred further development of the present invention relates to a process for carrying out thermal rearrangement reactions in a thin film evaporator under distillative conditions for the production of the odorant 3-(4-isopropylcyclohexen-1-yl) propanal.

Surprisingly, it was found in the context of the present invention that when thermal rearrangement reactions are carried out in a thin film evaporator, the selectivity of thermal rearrangement reactions can be increased, which in turn results in higher yields and increased purity of the chemical products due to the milder reaction conditions. It is known that at higher temperatures, i.e. higher thermal energy, the selectivity of chemical reactions decreases. Conversely, this means that at low temperatures, i.e. lower thermal energy, the selectivity increases, whereas at high temperatures an almost uniform product distribution can be expected. However, it should be noted that chemical reactions can often proceed more slowly at lower temperatures, which may result in longer production times. It has been shown that the process according to the present invention represents an optimized balance between mild reaction conditions and efficient conversion of the reaction educts and therefore offers an optimal compromise between yield, purity, and productivity.

However, the mild conditions of the present invention relate not only to the temperatures as such, but also to the residence times of the reaction mixture on the heated inner surface of the evaporator. The process according to the invention enables significantly shortened local residence times and thus reduces the risk of local thermal stress, in particular regarding already converted, i.e. rearranged reactants, and thus reduces thermal decomposition, i.e. thermal product damage. The process presented is therefore particularly suitable in connection with heat-sensitive/temperature-sensitive fragrances and odorants.

These and other aspects, features and advantages of the present invention will become apparent to the person skilled in the art from a study of the following detailed description and the patent claims. Any feature of one aspect of the invention may be used or substituted in another aspect of the invention. The examples contained in the present application describe the invention without limiting it.

Numerical examples given in the form “from x to y” include the specified values. If several preferred numerical ranges are specified in this format, all ranges resulting from the combination of the different end points are also included.

Advantageous further embodiments and variants of the invention are given in the dependent claims.

### DETAILED DESCRIPTION OF THE INVENTION

A first object of the present invention relates to a process for preparing a fragrance or odorant by carrying out thermal rearrangement reactions comprising the following steps:
a) Providing at least one reaction educt and/or other reagents in a thin film evaporator;
b) Carrying out at least one thermal rearrangement reaction on the reaction educts from step a) in the thin film evaporator under distillative conditions;
c) Obtaining the fragrance or odorant.

In the process for carrying out thermal rearrangement reactions, at least one reaction educt is provided in a first step a).

Depending on whether said thermal rearrangement reaction takes place in the presence of further reagents, such as catalysts, further corresponding reagents are also provided in step a) of the method according to the invention.

A preferred embodiment of the present invention relates to thermal rearrangement reactions which require acidic conditions and/or take place under acid catalysis. In said case, step a) of the present invention comprises providing at least one reaction educt and/or at least one acid in a thin film evaporator.

Suitable acids in this context are, for example, organic acids and their acid salts, such as carboxylic acids, alcohols, phenols, enols, thiols, sulphuric acid esters and sulphonic acids, phosphoric acid esters and phosphonic acid, CH- and NH-acidic compounds and their salts. Also suitable are inorganic acids and their acidic salts, such as hydrochloric acid, carbonic acid, phosphoric acid, nitric acid, nitrous acid, sulphuric acid, sulphurous acid, thiosulphuric acid and their salts. However, the use of organic acids and their salts in step a) is particularly preferred.

Preferably, aliphatic carboxylic acids, substituted carboxylic acids, heterocyclic carboxylic acids and aromatic carboxylic acids and their salts (carboxylates) are used in the process described herein. Compounds in which the OH group of the carboxy group is replaced by another group, e.g. —OR, —NH₂ or —Cl (carboxylic acid derivatives) such as carboxylic acid esters, carboxylic acid amides and carboxylic acid halides and their salts are also suitable acids in the context of the present invention.

These preferably have one or more carboxy groups (—COOH). Suitable carboxylic acids include: Acetic acid, acrylic acid, oxalic acid, formic acid, trifluoroacetic acid, succinic acid, fumaric acid, maleic acid, trichloroacetic acid, citric acid, aromatic carboxylic acids such as acetylsalicylic acid, benzoic acid, phenylacetic acid and salicylic acid, amino acids such as alanine, aspartic acid and glycine, as well as the salts of the aforementioned acids.

Particularly preferred acids in this context are selected from the group consisting of: organic acids and their acidic salts, with particular preference being given to carboxylic acids with one or more carboxy groups and the acid salts of the aforementioned compounds].

In a further preferred embodiment of the present invention, the use and provision of salicylic acid in step a) is particularly preferred.

The term “reaction educt” is understood to mean that the reactants used in the process according to the invention represent the starting material of the thermally induced rearrangement reaction. However, these themselves may have been produced from other educts in further preceding steps.

Step b) of the process described herein according to the invention comprises carrying out at least one thermal rearrangement reaction on the reaction educts from step a) in the thin film evaporator under distillative conditions.

Thermal rearrangement reactions concern all those chemical reactions in which, from a structural point of view, new chemical compounds are formed by displacements of individual atoms or groups of atoms accompanied by bond cleavages and new bond formations within a molecule, i.e. intramolecularly. Thermal rearrangements are initiated by means of thermal energy, i.e. thermal energy in the form of heat must be supplied for the thermal rearrangement reaction to take place.

The reaction educts described herein are therefore preferably chemical compounds which, after undergoing thermal rearrangement reactions, exhibit interesting and surprising properties. Such products can therefore preferably be fragrances or odorants and flavorings, such as the compound 3-(4-isopropylcyclohexen-1-yl) propanal of formula (I), which is used as a lily of the valley fragrance. Therefore, in a preferred embodiment, the present invention relates to a process for carrying out thermal rearrangement reactions for the alternative production of fragrances and flavorants, preferably the fragrance 3-(4-isopropylcyclohexen-1-yl) propanal.

In principle, however, all compounds that can undergo thermal rearrangement reactions are conceivable as reaction educts.

If several reaction educts or reagents are involved in the rearrangement, it is advantageous to feed them to the thin-film evaporator in a homogenized manner.

At this point, i.e. in process step a), no conversion usually takes place, as the thermal activation energy required for the thermal rearrangement process must first be added to the reaction educt mixture. This takes place preferably after feeding into the thin film evaporator, only inside the thin film evaporator.

In the context of the present invention, a thin-film evaporator is understood to be an apparatus for the distillative separation of reaction mixtures, in which the reaction mixture to be separated is distributed uniformly and in a defined thin film on the heated inner surfaces of the evaporator by means of specially designed wipers. Vertically constructed thin-film evaporators are particularly preferred, designed with an internal device for distributing the reaction mixture and an external device for heating the inner surfaces of the evaporator. The prepared reaction mixture comprising the at least one reaction educt and/or other reagents is preferably fed into the upper part of the thin-film evaporator and then distributed as a thin film onto the heated evaporator inner surface by means of rotating wiper elements.

Therefore, the thin film evaporator used in the present process is preferably equipped with a rotating wiper system. The choice of the appropriate wiper system depends on the properties of the reaction mixture, such as its viscosity.

The thin-film evaporator used herein has a cylindrical interior whose evaporator inner surfaces can be heated to a defined temperature, a dosing pump on the upper part of the stirring blades designed to feed in the reaction mixture, a rotating wiper system with adjustable rotation speeds and a vacuum pump to adjust the pressure, a collecting glass flask at the lower end and a cooling and head removal system for the vapors.

In the present invention, a thermal rearrangement of the at least one reaction educt and/or further reagents therefore takes place on the thin film evaporator, i.e. on the evaporator inner surfaces of the thin film evaporator. In such a reaction, reaction by-products and or unreacted reaction educts are removed from the reaction mixture directly in the thin film evaporator by means of distillation.

The phrase “under distillative conditions” therefore refers to the separation of certain reactants (reaction products or reaction educts) from the reaction mixture by thermal evaporation and subsequent reliquefaction in the classic sense. Separation of substances by evaporation and subsequent condensation is therefore a thermal separation process.

The simultaneous separation of reaction by-products and reaction products, which are enriched in the sump inside the receiving flask, enables a continuous shift of the reaction equilibrium to the product side, which allows the reaction educts to be converted as completely as possible.

Such processes can therefore generally be classified as reactive distillations, since a reaction, in this case the thermal rearrangement, and a separation process, namely the distillative separation of the by-products formed during the rearrangement, are combined in one process step. The process according to the invention comprising a reactive distillation is therefore carried out on a thin film evaporator.

The light ends consisting of reaction by-products and/or unreacted reaction educts are thus distilled off at the top of the column and then condensed, while the product is enriched in the sump inside the receiving flask. Among other things, this leads to a concentration of the product. The distilled compounds can be further purified in additional fine distillation steps and are then available for reuse as reaction educts or reagents in other manufacturing processes.

In a further preferred embodiment of the present process, the thermal rearrangement reaction takes place under distillative conditions under additional inert conditions.

In an alternative embodiment of this, an additional stripping process can be provided.

The use of thin film evaporators as a reaction vessel for the production of fragrance or odiferous substances offers the possibility of uniformly evaporating the reaction mixtures contained therein in the form of a thin film. Since the thin film evaporator in the present invention is preferably operated in a vacuum, the process described herein enables the use of lower temperatures and is therefore suitable for the gentle thermal rearrangement and simultaneous separation of by-products and thus makes it possible to simultaneously combine the conversion of the educts and the separation and purification of the products in a single process step.

Furthermore, the constantly rotating wiper blades of the thin film evaporator ensure that the reaction educts are evenly distributed, so that the reaction equilibrium is already fully established when they flow through the interior of the thin film evaporator. Furthermore, shorter residence times of the reactants at the heating surfaces and simultaneously high evaporation rates are achieved, making the process described herein particularly suitable for the conversion of temperature-sensitive reaction reactants. These reduced thermal stresses on the reaction educts used, but also on the reaction products, in particular due to the shortened residence times and avoided local overheating of the reactants compared to conventional flask experiments, characterize the process according to the invention as a very gentle process which reduces unwanted thermal decomposition.

The good mixing effect in conjunction with the low thickness of the film results in maximum evaporation rates and minimized residence times in the evaporator at moderate temperatures, which enables the production of quantitative and high-quality (temperature-sensitive) fragrances and odiferous substances.

The operating settings of the thin film evaporator, in particular the choice of temperature, pressure, liquid flow rate of the reaction mixture and wiping speed, i.e. the rotation speed of the wiper system, depend largely on the type of thermal rearrangement reaction and the reaction educts used and are adapted to these accordingly in order to minimize the typical residence times of the chemical components on the evaporation surface, i.e. on the heating surface, and to ensure gentle conversion and separation.

In the present invention, a gentle conversion or separation thus means that the thermal stress and the residence time during rearrangement and/or distillation are low and the corresponding reaction product does not suffer any negative thermal damage, which can be noticeable in a thermal decomposition or a change in the product properties such as color, odor, stability, etc.

The resulting fast reactions and associated short residence times enable an increase in the space-time yield and at the same time reduce the thermal stress on the reactants. Rapid separation of the reaction product formed from the reaction mixture enables higher yields. Furthermore, the process described herein is characterized by its low complexity compared to multi-stage processes. Thus, the process described herein is characterized as a technically and economically advantageous process, which is particularly suitable as a large-scale process due to the high space-time yield and simplicity of implementation.

A further advantage of the method described herein is that thermal rearrangement reactions, which require high temperatures to activate the rearrangement process, can be realized by applying a vacuum to the thin film evaporator using moderate temperatures by applying a corresponding vacuum to the thin film evaporator, which has a positive effect on energy costs and reduces thermal decomposition.

Overall, it has thus been surprisingly shown that when carrying out thermal rearrangement reactions in a thin film evaporator according to the method of the invention, the selectivity of thermal rearrangement reactions is increased, and higher yields and an increased purity of the chemical products can be achieved.

The low equipment requirements associated with the process described should also be emphasized. In particular regarding the thin-film evaporator, which functions both as a reactor for the thermal rearrangement and simultaneously as a distillation apparatus and thus makes specific apparatus for carrying out the thermal rearrangement reaction on the one hand and a separate distillation apparatus for purification on the other hand unnecessary.

This eliminates the need for laborious conversions and transfers of the reaction product, which would be associated with a loss of yield. The time aspect must also be considered here. Time and equipment savings are therefore reflected in reduced production costs. The present process is therefore ideally suited for the large-scale production of chemical products via thermal rearrangements.

Reactive distillation is preferably carried out at an operating temperature of the thin film evaporator of preferably 160 to 260° C., preferably 180 to 240° C. and more preferably at an operating temperature of 230° C. This corresponds to an approximate reaction temperature of 220° C. inside the thin film evaporator.

The actual contact temperature of the reactants, on the other hand, is preferably 160 to 240° C., even more preferably from 180 to 220° C. The reaction temperature or contact temperature is particularly preferably 220° C.

Preferably, the residence time of the reactants on the inner surface of the thin film evaporator is only a few seconds, which is significantly shorter than in reactive distillation.

The pressure in the evaporator is preferably 500 to 1000 mbar, particularly preferably approx. 200 mbar, whereby the pressure to be used depends on the product to be produced.

The reaction product of the thermal rearrangement reaction in step c) of the process described herein is preferably obtained at the lower end of the thin film evaporator in a receiving flask. The product enriched there is obtained in high yield and high purity based on the high selectivity of the process described herein.

The compound 3-(4-isopropylcyclohexen-1-yl) propanal is primarily used as a fragrance with a lily of the valley note. Despite a large number of existing fragrances, there is still a general need for new fragrances in the perfume industry, which are an indispensable component in the fragrance industry and in the manufacture of cosmetics, personal care products, as well as detergents and cleaning agents, etc. In addition to the search for new fragrances, the optimization of manufacturing processes in terms of efficiency, resource conservation and environmental aspects is currently a primary challenge in order to meet the high demand and to be able to offer high-quality products and ingredients.

Currently used production processes for the fragrance 3-(4-isopropylcyclohexen-1-yl) propanal comprise a thermal rearrangement of the corresponding acetal with cleavage of a vinyl ether to the desired product. However, the current production processes show low selectivities and therefore provide only low yields and low purity. In addition, the processes described above are not suitable for material-efficient and cost-reducing production.

In a preferred embodiment of the present invention, the process described herein relates to an optimized, alternative process for producing 3-(4-isopropylcyclohexen-1-yl) propanal.

Further preferred is the use of the process according to the invention as an alternative production method for the provision of the popular fragrance and flavoring agent 3,7-dimethylocta-2,6-dienal, also known as citral.

For the purposes of this text, a fragrance or odiferous substance is any substance that is capable of being used to create an olfactory impression, i.e. to convey an olfactory impression, or to alter (modify or enhance) the olfactory perception of another substance. In order to be used for perfumery purposes, this substance should preferably not have any undesirable side effects, e.g. effects that are harmful to the health or the environment, or effects that impair the intended use of a product containing this odorant or fragrance.

As already mentioned at the beginning, fragrances often have very specific odor profiles. It is therefore particularly desirable to synthesize such products with high purity, i.e. free of impurities and as selectively as possible, since even small amounts of impurities can distort or adversely affect the characteristic odor impression and cause unpleasant secondary odors. Such impurities can also have a negative effect on the stability of the fragrances produced due to chemical interactions and cause unpleasant odor notes or change the underlying odor impression. The highest possible degree of purity of such compounds is therefore desirable.

Current manufacturing processes often produce fragrances with insufficient purity and therefore require further optimization. In addition, due to the high demand for fragrances and odiferous substances, more time-, material- and therefore cost-efficient processes are required to replace the currently complex, expensive, and therefore uneconomical production methods.

Although fragrances and odiferous substances, for example, can currently be freed from unwanted by-products, reaction educts or degradation products by means of distillation, e.g. in a thin-film evaporator on a large scale, as mentioned above, the current manufacturing processes are associated with high costs, low selectivities and low material efficiency.

Surprisingly, it has been shown that a process according to the invention is suitable for the selective and thus highly pure synthesis of odorants or fragrances. The synthesized fragrances or odiferous substances therefore have a stable odor profile without secondary odors and are therefore suitable for further processing into perfumed products or perfume oils.

The process according to the invention is therefore particularly suitable for the economical production of highly concentrated and pure fragrances and odiferous substances in high yields through the combination of gentle and selective synthesis with reduced thermal stress and simultaneous distillation and thus purification of the products.

A further preferred further development of the present invention relates to a process according to the first object of the invention, wherein the at least one thermal rearrangement reaction comprises at least one pericyclic reaction.

In this context, pericyclic reactions are rearrangement reactions that undergo a cyclic transition state through a concerted, i.e. simultaneous intramolecular shift of electrons, without radical or ionic intermediates occurring. Examples of such reactions include sigmatropic rearrangements, particularly of the Claisen or Cope type, cycloadditions, chelatropic reactions or electrocyclic reactions. In principle, pericyclic rearrangement reactions can be initiated either thermally or photochemically, with thermally driven rearrangement reactions being preferred in the present invention.

It has been shown that such rearrangements can be carried out particularly selectively using the present process, which is reflected in a high yield and high purity of the products. Therefore, the present process is particularly suitable for carrying out pericyclic rearrangements in connection with the production of odiferous substances or fragrances.

As previously explained with regard to the first object of the invention, the selectivity of the thermal rearrangement reactions results in particular from the mild process conditions.

An alternative variant of the first object of the invention relates to a process for carrying out at least one rearrangement of the Claisen rearrangement, Cope rearrangement and/or intramolecular Prins reaction type.

With regard to sigmatropic rearrangement reactions, Claisen rearrangements and Cope rearrangements are of particular interest. These reactions are used alone or in combination in the synthesis of well-known fragrance compounds such as citral, a mixture of the cis-trans isomers geranial and neral.

In a known synthesis of the fragrance, the corresponding acetal is first cleaved and then rearranged according to Claisen and Cope. EP 0992477 B1 describes a continuous distillation process for the synthesis of citral. According to WO 2008037693 A1, the synthesis takes place in a distillation column, which is used as a reaction column, whereby the vapors are returned to the column in gaseous form.

Surprisingly, it has been shown that such reactions and syntheses can be carried out efficiently in a thin-film evaporator. The reaction educts are not converted in the sump of the column, but in a fine film along the inner surface of the thin-film evaporator. In contrast to the flask experiments described above, constant circulation of the reaction mixture through the wiper system prevents local overheating and reduces the thermal stress on the reactants as far as possible.

The acid-catalyzed carbonyl-ene reaction, also known as the Prins reaction, belongs to the group of cycloadditions. An intramolecular Prins reaction, i.e. an intramolecular carbonyl-ene reaction, is used, for example, in the synthesis of the fragrance isopulegol from citronellal.

Surprisingly, it was found that the odorant isopulegol could also be produced in high yield and high purity by means of the process described herein based on carrying out thermal rearrangement reactions in a thin film evaporator under distillative conditions.

As can be seen from this, the production of a large number of odorants or fragrances can be traced back to pericyclic rearrangement reactions of the Claisen, Cope and Prins type.

Surprisingly, it has been shown that these rearrangements, individually or in combination, are particularly selective according to the present process. The resulting products exhibit a high yield and high purity. Therefore, the present process is particularly suitable for carrying out at least one rearrangement according to Claisen, according to Cope and/or Prins individually or in combinations thereof, in particular for the production of odiferous substances or fragrances.

In a preferred embodiment of the present invention, the method according to the invention for carrying out thermal rearrangement reactions relates to carrying out rearrangements according to Claisen.

In a preferred embodiment of the present invention, the method according to the invention for carrying out thermal rearrangement reactions relates to carrying out rearrangements according to Cope.

Sigmatropic rearrangements are a special form of pericyclic reactions and are characterized by the displacement of σ bonds. The number of σ and π bonds remains the same before and after the rearrangement.

Oxy-Cope rearrangements require less activation energy than Cope rearrangements and therefore take place at lower temperatures. Such Claisen reactions (oxy-Cope rearrangements) are therefore, like the well-known Cope rearrangement [3,3]-sigmatropic rearrangements in which a carbon atom is replaced by an oxygen atom.

Successive rearrangement reactions, so-called tandem reactions, are intramolecular reaction sequences of rearrangements that occur spontaneously or in a targeted manner one after the other. Often, sigmatropic rearrangements preferably take place one after the other under constant reaction conditions and are therefore particularly suitable for implementation according to the present method in a thin-film evaporator.

As shown in examples 2 and 7 (citral), the method described herein is suitable for carrying out both individual [3,3]-sigmatropic rearrangements and several [3,3]-sigmatropic rearrangements in succession.

Tandem rearrangements are particularly preferred, in the order Claisen-Cope.

Therefore, in a next further development of the first object of the invention, the present invention relates to a process wherein at least one [3,3]-sigmatropic rearrangement is carried out.

Surprisingly, it has been shown that these [3,3]-sigmatropic rearrangements occur individually or in combination particularly selectively according to the present method. The resulting products can be isolated in high yields and with high purity.

In a further preferred embodiment of the present invention, the method according to the invention for carrying out thermal rearrangement reactions therefore relates to carrying out coupled Claisen and Cope rearrangements in all possible combinations.

Thus, in a preferred embodiment, the present invention describes a technological optimization of the Claisen rearrangement according to the invention based on an improved distillation process under reduced thermal stress. In formal terms, this improvement in the apparatus and thus in the process technology enables the solvent-free implementation in two chemical reaction steps, on the one hand the thermal rearrangement and on the other hand the purification by distillation, under the conditions of a reactive distillation within a short time and at low cost.

A further preferred variant of the first aspect describes a method according to the invention, further comprising in step a) providing at least one acid as reagent.

Particularly preferably used acids are selected from the group consisting of: organic acids and preferably aliphatic carboxylic acids, substituted carboxylic acids, heterocyclic carboxylic acids and aromatic carboxylic acids and their acid salts, whereby aromatic carboxylic acids are particularly preferable and in particular salicylic acid.

Many rearrangement reactions can be accelerated or carried out more selectively by using such catalysts.

However, the conversion of the reaction educts to the reaction products can in principle also be carried out without a catalyst, i.e. only by heating. However, the presence of an acid catalyst as described herein is particularly preferred and advantageous. The aforementioned acids and their acidic salts are therefore particularly suitable as acidic catalysts, with organic acids and their salts preferably being used.

Preferably, the acid is added as a reagent to the reaction educts and then the homogeneous reaction mixture is fed from a storage vessel via a dosing pump on the upper part of the stirring blades of the thin film evaporator.

Thermal rearrangement reactions, such as Claisen-type rearrangements, often only take place at very high temperatures. By using acidic catalysis, these reactions can be carried out at correspondingly milder temperatures, although strong acids such as phosphoric acid or Lewis acids are usually used for this purpose.

The preferred acids described herein are characterized by their good solubility in organic systems and their low tendency to form corrosion and are therefore particularly suitable for use in the present, gentler process within a thin film evaporator.

It has been shown that the already mild process conditions could be further mitigated, which leads to an even gentler treatment of the reactants, and in particular the reaction products. This means that the rearrangements to the desired reaction products described herein can be carried out cost-effectively, without great energy input and with high selectivities.

Particularly preferred is the provision of salicylic acid as a reagent in step a) of the process according to the invention for carrying out thermal rearrangement reactions in a thin-film evaporator under distillative conditions.

It was also observed that the use of acids selected from the list consisting of: organic acids and preferably aliphatic carboxylic acids, substituted carboxylic acids, heterocyclic carboxylic acids and aromatic carboxylic acids and their acidic salts, increases the selectivity of the rearrangements, and in particular of Claisen rearrangements, so that hardly any by-products are produced when carrying out the rearrangements. As a result, significantly improved chemical conditions for carrying out Claisen rearrangements were found and an increase in the selectivity of the rearrangement from approx. 70% to over 96% was observed.

Such an increase in selectivity makes a further decisive contribution to increasing the efficiency of the manufacturing process.

Particularly selective and therefore efficient conversions could therefore be observed in connection with Claisen rearrangements. The particularly gentle reaction conditions as well as the use of acids as described herein lead to very pure products in high yields.

A preferred embodiment of the present invention relates to carrying out thermal rearrangement reactions comprising at least one Claisen-type rearrangement, wherein the reaction is carried out using salicylic acid.

A process according to the first aspect and the preceding embodiments or variants, which further comprises, in step a), providing salicylic acid as a reagent for Claisen-type thermal rearrangements, thus represents a further preferred embodiment of the present invention.

The amount of acid used must be determined according to the product to be produced. Preferably, however, only catalytic amounts are used. These quantities vary, for example, from 1 mol % to 10% and are preferably around 5 mol %.

In a next variant of the process described above, the product of step c) is the compound 3-(4-isopropylcyclohexen-1-yl) propanal of formula (I):

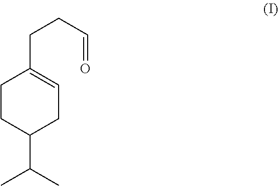

and their stereoisomers or mixtures thereof. The stereoisomers, in particular enantiomers and diastereomers, of the compound of the formula (I) individually or in mixtures are also particularly preferred.

As will be explained in more detail below, the compound of formula (I) can be synthesized by a process comprising at least steps a), b) and c) as described herein. For this purpose, the corresponding acetals are assumed to be reaction educts, these acetals being provided via an acid-catalyzed addition of a suitable alyl alcohol to an allyl vinyl ether.

Syntheses of the compound 3-(4-isopropylcyclohexen-1-yl) propanal of formula (I) are already described in EP 2578671 A1 and comprise the following steps:
i) By reacting a corresponding allyl alcohol with a corresponding alkyl vinyl ether in the presence of a protic acid such as phosphoric acid, the corresponding acetal of formula (II) is prepared.
ii) By reacting the acetal with catalytic amounts of acid such as hexanoic acid in a high-boiling solvent, the desired aldehyde of formula (I) is obtained via a vinyl ether as an intermediate.

However, the low selectivity of the Claisen rearrangement described is problematic, as 5-isopropyl-2-methylenecyclohexanol (III) is also formed during the acidic cleavage of the acetal (II).

This is illustrated in the following reaction scheme:

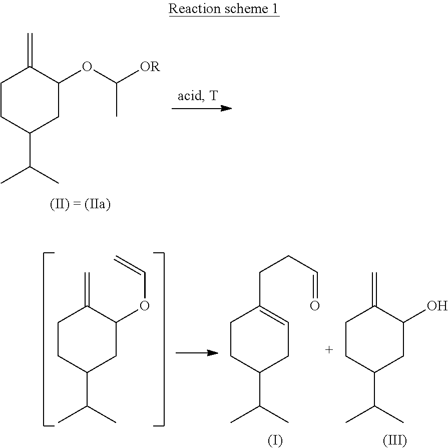

The use of salicylic acid as a catalyst for the present Claisen rearrangement leads to a significantly higher selectivity of the reaction, so that the formation of 5-isopropyl-2-methylenecyclohexanol (III) is largely suppressed during the acidic cleavage of the acetal (II), and the product is obtained in a high yield with high purity, so that the economic efficiency of the production can be increased.

It is known that odorants of the compound 3-(4-isopropylcyclohexen-1-yl) propanal of formula (I) have thermally labile properties. Short residence times and the associated reduced thermal stresses, as described herein, make it possible to counteract thermal decomposition processes and ensure high product quality. This avoids undesirable thermal decomposition products, which can interact adversely with the products or cause unpleasant secondary odors or distort or adversely affect the characteristic odor impression or have a detrimental effect on the stability of the fragrances.

The process according to the invention thus makes it possible to synthesize products that are as pure and free of impurities as possible, i.e. as selectively as possible, and are therefore particularly suitable for the production of high-quality and stable fragrances or odiferous substances.

Example 8 of the disclosure EP 2578671 A1 concerning the synthesis of 3-[(4R)-4-isopropylcyclohexen-1-yl]propanal shows that the process described therein only provides the desired product in a purity of 52%. This corresponds to 58% of the theoretically possible yield. Only further purification yields a purity of 96.5%. Therefore, after the second purification, a significantly lower yield of around 30% can be expected. It should also be noted that the hexanoic acid used therein is classified as toxic.

As can be seen from Example 2 of the present invention, a significantly purer product with a purity of 72% is obtained directly after synthesis. Subsequent purification finally yields an even purer product with 98.2% purity.

Thus, it can be observed that a process according to the present invention provides significantly purer products on a larger scale compared to conventional manufacturing processes. In addition, the use of toxic chemicals is replaced by significantly less hazardous substances.

A direct comparison of distillation processes shows that the process according to the invention operates under significantly milder conditions (comparison of examples 2 and 3) and furthermore that the use of environmentally hazardous, air- and light-sensitive substances such as trioctylamine and dibenzyl ether can be avoided.

The process described herein runs according to the following reaction scheme (path A and/or path B):

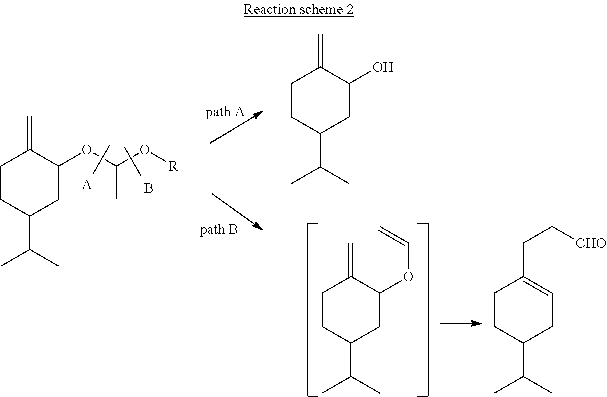

The product obtained from the reaction according to “path A” corresponds to a recovery of the educt, which can be reacted again. Based on the present process, potentially occurring by-products can thus be efficiently reused and thus high yields, high purities, high selectivities and low amounts of undesired by-products can be achieved.

Based on the gentle conversion due to low thermal stress, selectivity and purity can be maximized without sacrificing yield.

Furthermore, it can be stated that the mild reaction conditions of the process according to the invention are to be preferred due to the low energy input and result in controllable and uniform selectivities. Short residence times and the use of acid catalysts enable an increase in the space-time yield and reduce the thermal stress on the chemical products, which often has a negative effect on the quality of the products, for example due to thermal decomposition.

In addition, it can be stated that, compared to example 3, significantly fewer reactants are involved in the process, the need for chemicals is lower and thus a significant saving in terms of time, costs and resources can be achieved with the processes according to the invention.

It could therefore be shown that the present process is significantly more material-efficient than conventional manufacturing processes and other distillation processes such as reactive distillation, as shown in the examples described herein.

In another further embodiment of the process according to the invention, the reaction educts are selected from the group comprising the acetals of formulae (IIa), (IIb) and/or (IIc):

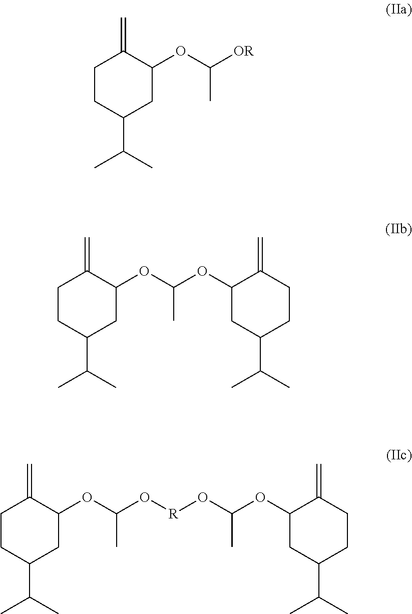

wherein the radical R in the compounds of the formula (IIa) represents linear or branched alkyl groups C₁₋₂₀, benzyl groups, acetyl groups phenyl groups, —CH₂—CH₂—OCH₃, —CH(CH₃)CH₂—OCH₃, —(CH₂)₂—O—(CH₂)₂—O—CH═CH₂, —(CH₂)₂—O—(CH₂)₂—O—(CH₂)₂—O—CH═CH₂ or 2-methylene-tetrahydrofuran, and in compounds of the formula (IIc) represents linear or branched alkyl groups C₁₋₂₀, —(CH₂)₂—O—(CH₂)₂—, or —(CH₂)₂—O—(CH₂)₂—O—(CH₂)₂—, as well as their stereoisomers, in particular diastereomers and enantiomers, and mixtures thereof.

The compounds of formula (IIa), formula (IIb) or formula (IIc) may be present in various forms corresponding to the possible constitutional isomers (regioisomers) for the radicals R, as well as stereoisomers, in particular enantiomers, diastereomers, of formula (IIa), formula (IIb) or formula (IIc), and as mixtures of the stereoisomers in any desired mixing ratio.

Surprisingly, the use of the thin film evaporator as a reactor has been shown to enable the production of 3-(4-isopropylcyclohexen-1-yl) propanal (I), starting from acetals of the form (IIa), (IIb) or (IIc).

By functionalizing the side chains with the preferred residues described herein, a further increase in selectivity was observed during the rearrangement of the acetals to the desired products.

Particularly strong increases in selectivity were observed in connection with the acid catalysis described herein during the Claisen rearrangement.

Particularly high increases in selectivity and thus higher purity and yield were observed with regard to ethyl groups, butyl groups and —(CH₂)₂—O—(CH₂)₂—O—CH═CH₂ as radicals R.

An alternative further development of the process according to the invention therefore relates to the reaction educts of formula (IIa), wherein the radicals R of the acetals of formula (IIa) are preferably ethyl groups, butyl groups and —(CH₂)₂—O—(CH₂)₂—O—CH═CH₂.

In a preferred embodiment, the radical R of the acetals of formula (IIa) is in particular preferably-(CH₂)₂—O—(CH₂)₂—O—CH═CH₂.

The aforementioned embodiment shows the greatest selectivity-enhancing effect and is therefore particularly preferred in the context of the present invention.

In a next variant, the present invention relates to a process, further comprising an additional/preceding process step 0) prior to step a), wherein in this step 0) the reaction educts of formulae (IIa), (IIb) and/or (IIc) are provided via acid-catalyzed addition of 5-isopropyl-2-methylenecyclohexanol (formula (III)) to the corresponding vinyl ethers of formula (IV):

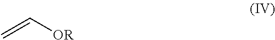

wherein the radical R represents a linear or branched alkyl group C₁₋₂₀, benzyl groups, acetyl groups, phenyl groups, —CH₂—CH₂—OCH₃, —CH(CH₃)—CH₂—OCH₃, (CH₂)₂—O—(CH₂)₂—O—CH═CH₂, (CH₂)₂—O—(CH₂)₂—O—(CH₂)₂—O—CH═CH₂ or 2-methylene tetrahydrofuran.

Step 0) of the process according to the invention therefore relates to a way of preparing the reaction educts according to the invention, i.e. the corresponding acetals of formula (II) as described herein, by acidic reaction of the known compound 5-isopropyl-2-methylenecyclohexanol of formula (III) with vinyl ethers of formula (IV).

The resulting acetals (II) are cleaved into the corresponding vinyl ethers using acid-catalysis, which are directly thermally converted into the desired aldehyde 3-(4-isopropylcyclohexen-1-yl) propanal of formula (I) by Claisen rearrangement.

As described herein, the conversion of acetals of formulae (IIa), (IIb) and (IIc) as described herein to 3-(4-isopropylcyclohexen-1-yl) propanal of formula (I) according to the process according to the invention is significantly more selective than prior art processes, whereby significantly higher yields and purer products can be obtained. Nevertheless, a certain small proportion of by-products such as the 5-isopropyl-2-methylene-cyclohexanol of formula (III) described herein may be obtained according to reaction scheme 1. The combination of the process of thermal conversion described herein and simultaneous purification of the reactants by distillation makes it possible to collect such a by-product as a result of the distillation process in largely pure form and then to reuse it in step 0) for the production of the reaction educts of formulae (IIa), (IIb) and (IIc) mentioned and thus ensures a material-efficient use of the raw materials used.

This thus underlines the environmentally friendly, resource-saving and cost-saving nature of the process according to the invention.

According to another further development of the process according to the invention, Lewis acids or Brønstedt acids are preferably used as acid catalysts for the acid-catalyzed addition in step 0).

Lewis acids and Brønstedt acids are preferably used as acids for the acid-catalyzed addition. In particular, magnesium salts such as magnesium (II) chloride, magnesium (II) sulphate, ammonium sulphate, phosphoric acid and zeolites are preferred.

The acids used as catalysts herein lead to an increased selectivity of the addition reaction.

In a further variant of the present invention and thus also of the process according to the invention, for the acid-catalyzed addition of 5-isopropyl-2-methylenecyclohexanol (formula (III)) to the corresponding vinyl ethers of formula (IV) in step 0) of the process according to the invention, the corresponding vinyl ethers of formula (IV) are used in a 1.1 to 5.0-fold molar excess.

The use of the vinyl ethers of formula (IV) in a defined molar excess thereby influences the selectivity of the formation of the acetals, i.e. the reaction educts of formulae (IIa) and (IIc).

In particular, the use of vinyl ethers of formula (IV) in a 1.2 to 2.0-fold molar excess is preferable.

For the use of the vinyl ethers of formula (IV) in such an excess, the greatest selectivity-enhancing effects of the addition reaction to the reaction educts described herein can be observed.

In this context, it is particularly preferable to recover the vinyl ether of formula (IV) remaining in the reaction mixture by distillation.

Unreacted or excess vinyl ether of formula (IV) from step 0) of the present process is preferably recovered by distillation. Distillation in a vacuum is particularly preferred.

Vacuum distillations require lower temperatures and are therefore used as a particularly gentle process using moderate temperatures.

Distillative reprocessing of the vinyl ether of formula (IV) from step 0) enables the vinyl ether to be used again in subsequent acid-catalyzed reactions with 5-isopropyl-2-methylenecyclohexanol of formula (III) for the preparation of further reaction educts of formulae (IIa), (IIb) and (IIc) or for other purposes.

This material-efficient use of raw materials makes a decisive contribution to the economic efficiency of the process described herein. Such reuse conserves the existing resources and makes a decisive contribution to a sustainable and environmentally friendly process, while at the same time minimizing production and disposal costs.

In a particularly preferred further development of the method described herein, the method runs as a continuous process.

Continuous processes are used in particular for processing large quantities of raw materials and are characterized by a continuous, i.e. uninterrupted process. Continuous processes make it possible to reduce the number of intermediate steps, such as refilling or cooling and heating the equipment, and are therefore more economical than batch processes.

Furthermore, continuous manufacturing processes guarantee a constant, i.e. consistent, product quality without quality fluctuations between individual batches.

In continuous processes, a constant flow equilibrium is established. The equilibrium of the reaction is shifted towards higher yields by constant separation of the products. Furthermore, a continuous separation of unreacted educts or by-products enables a higher purity of the products to be achieved due to the distillative character of the present invention, as the probability of possible side reactions is thus significantly reduced.

Overall, continuous processes have a clear economic advantage over batch processes and are particularly suitable for manufacturing processes on industrial scale and are therefore particularly preferred in the context of the present invention.

In order to carry out the thermal rearrangement reactions in a continuous or semi-continuous process, the thin film evaporator described herein can be expanded into a circulation reactor. For this purpose, reaction educts are pumped back into the storage vessel and fed back into the upper part of the stirring blades of the thin film evaporator via the dosing pump. This leads to an extension of the reaction time with a simultaneously shortened residence time, so that a complete conversion of the reaction educts can be guaranteed.

In particular, it is preferable to subject the distilled substances to a distillative separation process again before they are fed back into the system. For this purpose, it is advisable to use equipment suitable for fractional distillation and to return only the corresponding reaction educts to the thin film evaporator.

In this way, unreacted reaction educts can be returned to the manufacturing process in order to reduce the amount of environmentally harmful reaction by-products and at the same time ensure material and cost-efficient use of raw materials.

It should also be emphasized that continuous processes are usually more stable and uniform due to the constant reaction conditions and thus ensure higher selectivities of the reactions, which can increase the purity and yield of the products.

Furthermore, a preferred further development of the present invention describes a process comprising the recovery of 5-isopropyl-2-methylenecyclohexanol of formula (III), other starting compounds and/or non-rearranged reaction educts of the process.

In the course of the reactive distillation according to the invention, in which the product is enriched in the receiving flask, unreacted reaction educts or precursors and by-products can be removed by distillation and then returned to the process.

In particular, the recovery of the compound 5-isopropyl-2-methylenecyclohexanol of formula (III) is preferred. This compound, which may occur as a possible by-product despite the increase in selectivity of the reaction described herein, can subsequently be reused for the production of the reaction educts in step 0) of the process.

This conserves resources and at the same time saves costs for disposal and repurchasing of the same compound.

The preferential recovery of raw materials described herein enables an optimized and efficient use of materials in the production, taking into account environmental aspects and enables a reduction of the manufacturing costs incurred.

Finally, in a second aspect, the present invention relates to the product directly produced by the process according to the invention as described herein.

As explained above, the process described herein provides products with excellent purity in high yield based on an optimized and highly selective manufacturing process under distillative conditions. At the same time, the products described herein can be produced on a large scale in a cost-effective and environmentally friendly manner.

The high efficiency of the process and the resulting products enable to meet the high demand cost-effectively and completely.

In a preferred embodiment, the product is prepared directly from the process according to the invention as herein preferably the compound 3-(4-isopropylcyclohexen-1-yl) propanal of formula (I), which is used in particular as a fragrance with a lily of the valley scent.

### EXAMPLES

The present invention is described in more detail below with reference to examples of embodiments. First, the examples given relate to the preparation of compounds of formula (I) and particularly preferred compounds. Furthermore, it should be noted that the IUPAC nomenclature may differ from the generic designation used so far.

For spectroscopic data, the following English-language regulation applies regarding the use of dots as separators for numerical data in order to ensure better clarity of the measurement results. In this context, in data of the form “δ=7.12 (dd, J=3.7, 0.9 Hz, 2H)” the measured values are to be read as “δ=7.12”, “3.7” and “0.9”. The alternative point-comma rule is used for the NMR data.

In Examples 2 and 3 below, the synthesis of the compound 3-(4-isopropylcyclohexen-1-yl) propanal of formula (I) starting from the same educt 4-isopropyl-1-methylene-2-[1-[2-(2-vinyloxyethoxy) ethoxy]ethoxy]cyclo-hexane from Example 1 is described.

### Example 1: Acetal

### Synthesis of 4-isopropyl-1-methylene-2-[1-[2-(2-vinyloxyethoxy) ethoxy]ethoxy]-cyclohexane (Reaction Educt)

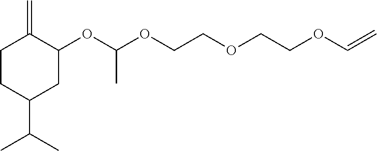 [SMILES: C=COCCOCCOC(C)OC1CC(C(C)C)CCC1=C]

In a 2 L three-neck reaction flask with dropping funnel, intensive condenser and magnetic stirrer, 500.00 g (2.91 mol, 89.8% purity) of 5-isopropyl-2-methylenecyclohexanol and 1.68 g (0.01 mol) of 85% phosphoric acid are introduced and heated to 30° C. Then 923.18 g (5.82 mol) diethyleneglycol divinyl ether is added in such a way that the temperature does not rise above 40° C. The reaction is stirred at the same temperature for a further 4 hours until complete conversion and then cooled to room temperature. The reaction mixture is then taken up in 1.3 L of tert-butyl methyl ether and stirred with 1.3 L of a saturated sodium carbonate solution for 20 min. The phases are separated and the organic phase is washed with 1.0 L of a saturated sodium carbonate solution. The aqueous phases are extracted once with 800 mL of tert-butyl methyl ether. The organic phases are combined and dried over sodium sulphate and then filtered. The volatile components, the solvent and excess diethylene glycol divinyl ether (Sdp. 60° C. at p=2.5 mbar) are finally distilled under vacuum. The 914.2 g residue obtained in this way is used directly in the subsequent reaction (examples 2 and 3) as the crude product 4-isopropyl-1-methylene-2-[1-[2-(2-vinyloxyethoxy) ethoxy]ethoxy]cyclohexane.

### Spectroscopic Data:

EI-MS m/z (%): 268 (1, [M−44]⁺), 225 (2), 180 (4), 162 (19), 137 (65), 115 (58), 110 (54), 93 (50), 87 (100), 81 (85), 68 (85), 55 (19), 43 (77), 29 (15).

¹H-NMR (400 MH2, CDCl₃, 300 K): δ=7.18-7.15 (m, 1H), 7.13 (dd, J=4.8, 1.9 Hz, 1H), 7.07 (d, J=1.7 Hz, 1H), 7.07-7.03 (m, 1H), 4.88 (s, 2H), 1.68 (s, 6H) ppm.

¹³C-NMR (101 MH2, CDCl₃, 300 K): δ=206.99, 192.78, 191.80, 191.67, 163.50, 133.88, 132.25, 132.13, 131.98, 130.04, 129.15, 128.86, 128.60, 128.56, 127.95, 127.26, 126.32, 125.51, 124.50, 123.52, 86.16, 82.02, 70.23, 64.25, 63.17, 63.15, 30.92, 29.47, 29.12 ppm.

### Example 2: DV Reaction

### Synthesis of 3-(4-Isopropylcyclohexen-1-Yl) Propanal of Formula (I) Using the Thin Film Evaporator According to the Method of the Invention

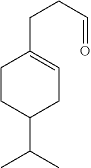

The reaction mixture is fed from a storage vessel via a dosing pump at the top of the stirring blades of the thin film evaporator. The thin film evaporator is equipped with cooling and head removal, adjustable setting of the rotor blades, a vacuum pump for setting the pressure and a collecting glass flask at the lower end.

170.00 g of a 5% aqueous sodium carbonate solution is placed in the receiving flask to buffer the pH value. The rotation speed is set to 500 rpm stirring speed and a vacuum of 800 mbar is applied. The thin film evaporator is heated to T=230° C. In the storage vessel, which is emptied via the dosing pump, 160.00 g of 4-isopropyl-1-methylene-2-[1-[2-(2-vinyloxyethoxy) ethoxy]ethoxy]cyclohexane from example 1 and 8.00 g (57.34 mmol, 5.0 m %) of salicylic acid are weighed in and stirred. The solution is applied to the thin film evaporator at a rate of 2.5 mL/min. During the reaction, light volatiles are distilled off at the top of the column. The product 3-(4-isopropylcyclohexen-1-yl) propanal of formula (I) is enriched in the sump inside the receiving flask. After completion of the reaction, the device is cooled down and depressurized. 150 mL of tert-butyl methyl ether are added to the reaction solution in the receiving flask which is then extracted. After phase separation, the aqueous phase is again extracted with 150 mL tert-butyl methyl ether and the phases are then separated. The combined organic phases are washed with 250 ml of a saturated sodium chloride solution. After phase separation, the organic phase is dried over sodium sulfate and then filtered. The solvent is then removed in vacuo. In this way, 119.04 g of crude product with a purity of 72% is obtained.

119.4 g (72% purity) of the crude 3-(4-isopropylcyclohexen-1-yl) propanal of the formula (I) is subjected to distillative purification on a canned column. This isolates 77.4 g of 3-(4-isopropylcyclohexen-1-yl) propanal of the formula (I) with a purity of 98.2% (74-75° C. at 1.0 mbar; R (reflux)/D (removal)=100/1).

The analytical data correspond to those in the literature (EP 2578671 A1).

### Example 3: Reactive Distillation

### Synthesis of 3-(4-Isopropylcyclohexen-1-Yl) Propanal of Formula (I) Under Reactive Distillation Conditions

In a 0.5 L three-necked reaction flask with Liebig condenser, small column and magnetic stirrer, 160 g (=0.509 mol feed) of 4-isopropyl-1-methylene-2-[1-[2-(2-vinyloxyethoxy)-ethoxy]ethoxy]cyclohexane from example 1 is weighed out and mixed with 0.67 g of lithium dihydrogen phosphate, 0.67 g of trioctylamine and 160 g of dibenzyl ether. The reaction mixture is then heated to 175° C. at 100 mbar and the light volatiles are distilled at the top of the column for 5 hours. After complete conversion, 3-(4-isopropylcyclohexen-1-yl) propanal of formula (I) (at 175° C., 1 mbar) is then distilled off from the bottom. This yields 275.1 g of the crude product with a purity of 24.3%.

Subsequently, 275.1 g (24.3%) of the crude 3-(4-isopropylcyclohexen-1-yl) propanal of the formula (I) is subjected to a distillative purification on a canned column. This isolates 61.5 g of 3-(4-isopropylcyclohexen-1-yl) propanal of the formula (I) with a purity of 97.8% (74-75° C. at 1.0 mbar; R/D=100/1).

The analytical data correspond to those in the literature (EP 2578671 A1).

### Example 4

### Synthesis of 4-isopropyl-2-[1-(2-methoxy-1-methyl-ethoxy) ethoxy]-1-methylene-cyclohexane (Reaction Educt)

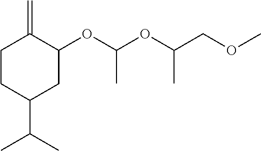 [SMILES: C=C1CCC(C(C)C)CC1OC(C)OC(C)COC]

44.66 g (260 mmol, 89.8% purity) of 5-isopropyl-2-methylene-cyclohexanol and 0.15 g (1.3 mmol) of 85% phosphoric acid are placed in a 250 mL three-necked reaction flask with a dropping funnel, intensive condenser and magnetic stirrer and heated to 30° C. Then 61.14 g (520 mol) of 1-methoxy-2-vinyloxypropane is added in such a way that the temperature does not rise above 40° C. The reaction is stirred at the same temperature for a further 2.5 hours until complete conversion and then cooled to room temperature. The reaction mixture is then taken up in 100 ml of tert-butyl methyl ether and stirred with 100 ml of a saturated sodium carbonate solution for 10 min. The phases are separated and the organic phase is washed with 100 ml of a saturated sodium carbonate solution. The aqueous phases are extracted once with 100 ml of tert-butyl methyl ether. The organic phases are combined and dried over sodium sulfate and then filtered. The volatile components, the solvent and excess 1-methoxy-2-vinyloxypropane are finally distilled in vacuo using a ball tube apparatus (Sdp. 76-80° C. at p=0.6 mbar). The 74.57 g residue obtained in this way is used directly in the subsequent reaction (example 5) as the crude product 4-isopropyl-2-[1-(2-methoxy-1-methyl-ethoxy) ethoxy]-1-methylene-cyclohexane (purity 88%).

The analytical data correspond to those in the literature (EP 2578671 A1).

### Example 5

### Synthesis of 3-(4-Isopropylcyclohexen-1-Yl) Propanal of Formula (I) Under Reactive Distillation Conditions

In a 250 mL three-neck reaction flask with Liebig condenser, 8 cm long Vigreux column and magnetic stirrer, 74.00 g 4-isopropyl-2-[1-(2-methoxy-1-methyl-ethoxy) ethoxy]-1-methylenecyclohexane (88% purity) from example 4 is weighed out and 311 mg lithium dihydrogen phosphate, 311 mg trioctylamine and 74 g (71.15 mL) dibenzyl ether are added. The reaction mixture is then heated to 175° C. at 430 mbar and the light volatiles are distilled at the top of the column for 3 hours. After complete conversion, 3-(4-isopropylcyclohexen-1-yl) propanal of formula (I) (124° C., 2.5 mbar) is then distilled off from the bottom. 100.22 g of 3-(4-isopropylcyclohexen-1-yl) propanal of the formula (I) is obtained in 27% purity.

The analytical data correspond to those in the literature (EP 2578671 A1).

### Example 6

### Synthesis of 3-methyl-1,1-bis(3-methylbut-2-enoxy) but-2-ene (Reaction Educt)

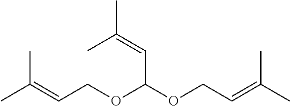 [SMILES: CC(C)=CCOC(C=C(C)C)OCC=C(C)C]

In a 4 L three-neck reaction flask with intensive condenser, magnetic stirrer and water separator, 1226 g (13.95 mol) of 3-methylbut-2-en-1-ol, 4.02 g (35 mmol) of 85% phosphoric acid and 400 g (4.65 mol) of 3-methylbut-2-enal are dissolved in 1.6 L of cyclohexane. The reaction mixture is then heated under reflux for 30 hours, the resulting water is separated off and the reaction mixture is then cooled to room temperature. The reaction mixture is then taken up in 500 mL of tert-butyl methyl ether and stirred in 800 mL of a 10% sodium carbonate solution for 10 min. The phases are separated and the organic phase is washed with 800 mL of a 5% sodium carbonate solution. The aqueous phases are extracted once with 500 ml of tert-butyl methyl ether. The organic phases are combined and dried over sodium sulphate and then filtered. The volatile components and the solvent are removed in vacuo. The crude product is fractionally distilled over a 12 cm Vigreux column (Sdp. 72-107° C. at p=1.2-2.2 mbar). The 537 g of 3-methyl-1,1-bis(3-methylbut-2-enoxy) but-2-ene (purity 79%, yield 53%) obtained in this way are used directly in the subsequent reaction (example 7).

The analytical data correspond to those in the literature.

### Example 7

### Synthesis of 3,7-Dimethylocta-2,6-Dienal Using the Thin Film Evaporator According to the Method of the Invention

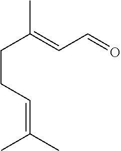

A reaction mixture is fed from a storage vessel via a dosing pump at the top of the stirring blades of the thin film evaporator. The thin film evaporator is equipped with cooling and head removal, adjustable setting of the rotor blades, a vacuum pump for setting the pressure and a collecting glass flask at the lower end.

The rotation speed is set to 500 rpm stirring speed. The thin film evaporator is heated to T=230° C. In the storage vessel, which is emptied via the dosing pump, 100.00 g of 3-methyl-1,1-bis(3-methylbut-2-enoxy) but-2-ene (79% purity, 331.7 mmol) from example 6 and 1.00 g (7.2 mmol, 1.0 m %) of salicylic acid are weighed in and stirred. The solution is applied to the thin film evaporator at a rate of 1.5 mL/min. During the reaction, light volatiles are distilled off at the top of the column. The product 3,7-dimethylocta-2,6-dienal is enriched in the sump inside the receiving flask. After completion of the reaction, the device is cooled down and depressurized. 100 ml of tert-butyl methyl ether are added to the reaction solution in the receiving flask and extracted with 70 mL of a saturated sodium hydrogen carbonate solution. After phase separation, the organic phase is washed again with 70 mL of a saturated sodium chloride solution. The phases are then separated, the organic phase is dried over sodium sulphate and then filtered. The solvent is then removed in a vacuum. In this way, 57.1 g of crude product is obtained. The crude product is then distilled using a ball tube apparatus (sump 75-117° C. at 0.6 mbar). This isolates 49.5 g of 3,7-dimethylocta-2,6-dienal with a purity of 74% (242 mmol, yield 73%).

The analytical data correspond to those in the literature.

A comparison of the methods shows that the desired products can be produced with a significantly higher purity using the process according to the invention (see Table 1). Therein, the proportion of undesirable by-products is significantly reduced, which indicates a more selective and at the same time gentler synthesis.

TABLE 1
Comparison of the reaction products from example 2 (according
to the invention) and example 3 (reactive distillation).
Reaction educt: Acetal from example 1 (educt for reaction):
914.2 g = 2.91 mol

| | Example 2 | Example 3 |
|---|---|---|
| | (according to | (reactive |
| | the invention) | distillation) |
| Feed | 457.1 g (50%) | 457.1 g (50%) |
| Crude | Yield: 119.4 g; | Yield: 275.06 g; |
| product | Purity: 72% | Purity: 24.31% |
| After fine | Yield: 77.4 g; | Yield: 61.5 g; |
| distillation | Purity: 98.2% | Purity: 97.8% |
| | Yield: | Yield: |
| | 221.1 g = 84.3% | 175.7 g = 66.9% |
| | of theory | of theory |

1. A process for the preparation of an odorant or fragrance by carrying out thermal rearrangement reactions comprising the following steps:
a) Providing at least one reaction educt and/or other reagents in a thin film evaporator;
b) Carrying out at least one thermal rearrangement reaction on the reaction educts from step a) in the thin film evaporator under distillative conditions;
c) Obtaining the odorant or fragrance.

2. The process according to claim 1, wherein the at least one thermal rearrangement reaction comprises at least one pericyclic reaction.

3. The process according to claim 1, comprising at least one rearrangement of the Claisen rearrangement type, the Cope rearrangement type and/or the Prins reaction type.

4. The process according to claim 1, comprising at least one [3,3]-sigmatropic rearrangement.

5. The process according to claim 1, further comprising in step a) providing at least one acid as a reagent.

6. The process according to claim 1, further comprising in step a), providing salicylic acid as a Claisen-type thermal rearrangement reagent.

7. The process according to claim 1, wherein the product of step c) is 3-(4-isopropylcyclohexen-1-yl) propanal of formula (I): 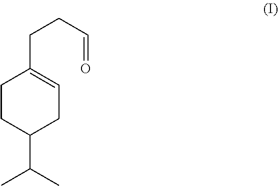
as well as its stereoisomers or mixtures thereof.

8. The process according to claim 7, wherein the reaction educts are selected from the group consisting of the acetals of formulae (IIa), (IIb) (IIc), and mixtures thereof: 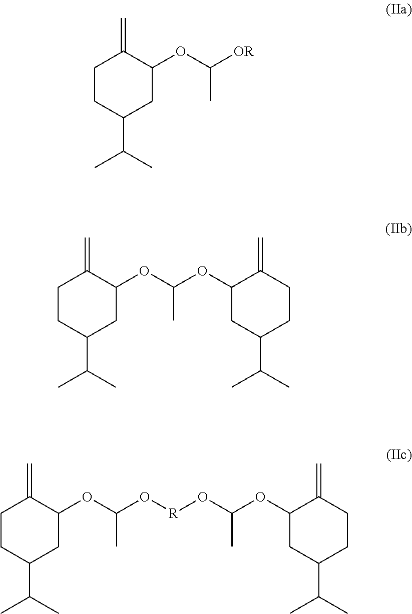
wherein R in compounds of the formula (IIa) represents linear or branched alkyl groups C₁₋₂₀, benzyl groups, acetyl groups, phenyl groups, —CH₂—CH₂—OCH₃, —CH(CH₃)—CH₂—OCH₃, —(CH₂)₂—O—(CH₂)₂—O—CH═CH₂, —(CH₂)₂—O—(CH₂)₂—O—(CH₂)₂—O—CH═CH₂ or 2-methylene-tetrahydrofuran, and in compounds of the formula (IIc) represents linear or branched alkyl groups C₁₋₂₀, —(CH₂)₂—O—(CH₂)₂—, or —(CH₂)₂—O—(CH₂)₂—O—(CH₂)₂—, as well as their stereoisomers, and mixtures thereof.

9. The process according to claim 8, wherein the radicals R of the acetals of formula (IIa) are ethyl groups, butyl groups or —(CH₂)₂—O—(CH₂)₂—O—CH═CH₂.

10. The process according to claim 8, further comprising a process step 0) prior to step a), wherein in step 0) the reaction educts of formulae (IIa), (IIb) and/or (IIc) are provided via acid-catalyzed addition of 5-isopropyl-2-methylenecyclohexanol to the corresponding vinyl ethers of formula (IV): 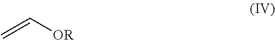
wherein R represents a linear or branched alkyl group C₁₋₂₀, benzyl groups, acetyl groups, phenyl groups, —CH₂—CH₂—OCH₃, —CH(CH₃)—CH₂—OCH₃, —(CH₂)₂—O—(CH₂)₂—O—CH═CH₂, —(CH₂)₂—O—(CH₂)₂—O—(CH₂)₂—O—CH—CH₂ or 2-methylene-tetrahydrofuran.

11. The process according to claim 10, wherein Lewis acids or Brønstedt acids are used as acid catalysts for the acid-catalyzed addition in step 0).

12. The process according to claim 10, wherein the vinyl ethers of formula (IV) are used in 1.1 to 5.0-fold molar excess.

13. The process according to claim 10, wherein the vinyl ether of formula (IV) remaining in the reaction mixture is recovered by distillation.

14. The process according to claim 1, wherein the process runs as a continuous process.

15. The process according to claim 8, further comprising the step of recovering 5-isopropyl-2-methylenecyclohexanol, other starting compounds and/or non-rearranged reaction educts.

16. The process of claim 8, wherein the stereoisomers of the acetals are diastereomers or enantiomers.
